(19) United States
(12) Patent Application Publication
(10) Pub. No.: US 2005/0054702 A1
(43) Pub. Date: Mar. 10, 2005

(54) Method of controlling allergens

(75) Inventors: Kelly A. Dunn, High Bridge, NJ (US); Patrick J. Lutz, Nazareth, PA (US)

Correspondence Address: DARBY & DARBY P.C., P. O. BOX 5257, NEW YORK, NY, 10150-5257, US

(73) Assignee: Lonza Inc., Fair Lawn, NJ (US)

(21) Appl. No.: 10/658,102

(22) Filed: Sep. 9, 2003

Related U.S. Application Data
(60) Provisional application No. 60/409,692.

(51) Int. Cl. A61K 31/4166; A61K 31/205

(52) U.S. Cl. 514/389; 514/554

(57) ABSTRACT

The present invention provides a method for controlling allergens by applying an anti-allergenic effective amount of one or more anti-allergenic agents selected from hyperbranched polymers, amines (including tertiary amines), hops extracts, anti-allergenic iodo derivatives, quaternary ammonium compounds, hydantoins and hydantoin blends, polyhexamethylenebiguanidine hydrochloride, natural oils, anti-allergenic natural acids, sodium carbonate, chelating agents, cyclic ketone acids (e.g., dehydroacetic acid (DHA) and salts thereof (including sodium dehydroacetate and the monohydrate thereof)), anti-allergenic surfactants, organic phosphates, hydrogen peroxide, butylene glycol, phenoxyethanol, sodium salicylate, and mixtures thereof, to the allergens or a substrate on which they are located. These anti-allergenic agents are particularly effective at controlling household allergens, such as dust mites.

This application claims the benefit of U.S. Provisional Application Ser. No. 60/409,692 filed Sep. 9, 2002, which is hereby incorporated by reference in its entirety.

### FIELD OF THE INVENTION

The present invention relates to a method of controlling allergens with certain anti-allergenic agents, such as tertiary amines and natural acids.

### BACKGROUND OF THE INVENTION

For over 15 million Americans, household allergens cause asthma, congestion, and general respiratory discomfort. Household dust contains many allergens attributed to dust mites, cockroaches, cats, dogs, and pollen. Various products have been developed that kill allergens such as dust mites; however, many of these products are prohibitively expensive. For instance, U.S. Pat. No. 5,672,362 discloses a method for controlling dust mites living on an inanimate surface by applying a particular aqueous disodium octaborate tetrahydrate solution onto the surface.

Furthermore, these products have many disadvantages, including high toxicity levels, biocidal activity limited to mites, and/or staining of light colored fabrics.

In recent years, new products have been developed which target dust mite allergens (e.g., mite debris) rather than the dust mites themselves. These products typically work by neutralizing, denaturing, or coating the allergens. For example, U.S. Pat. No. 4,048,369 teaches a method of controlling allergens by periodically coating various fabrics with a particular aqueous film forming solution. The coating reduces the allergens found in house dust by immobilizing mite debris within the fabric and maintaining the mite under less than optimum growth conditions.

German Patent Publication No. 20017213 discloses a disinfectant composition for the eradication of house dust mites and/or their eggs comprising an alkylpropylenediamine compound and Glucoprotamine™.

U.S. Patent Publication No. 2002/0040055 discloses the use of a rare earth metal salt to denature allergens.

There is a need for anti-allergenic agents which are less toxic and less expensive than those in existence, attack the allergen producing mite or cockroach, and neutralize household allergens produced by mites, cockroaches, cats, dogs, and pollen.

### SUMMARY OF THE INVENTION

The present invention provides a method for controlling allergens by applying an anti-allergenic effective amount of one or more anti-allergenic agents selected from hyperbranched polymers, amines (including tertiary amines), hops extracts, anti-allergenic iodo derivatives, quaternary ammonium compounds, hydantoins and hydantoin blends, polyhexamethylenebiguanidine hydrochloride, natural oils, anti-allergenic natural acids, sodium carbonate, chelating agents, cyclic ketone acids (such as those described in International Publication No. WO 02/069710, which is hereby incorporated by reference) (e.g., dehydroacetic acid (DHA) and salts thereof (including sodium dehydroacetate and the monohydrate thereof)), anti-allergenic surfactants, organic phosphates, hydrogen peroxide, butylene glycol, phenoxyethanol, sodium salicylate, and mixtures thereof, to the allergens or a substrate on which they are located. These anti-allergenic agents are particularly effective at controlling household allergens, such as dust mites. Without being bound by any particular theory, the inventors believe that many of the aforementioned anti-allergenic agents encapsulate and/or coat the allergens, thereby denaturing and/or deactivating them.

Preferred anti-allergenic agents include, but are not limited to:
(a) a tertiary amine having the formula NR¹R²R³ where R¹ is a C₁-C₁₈ alkyl (preferably a C₈-C₁₈ alkyl) and R² and R³ are —(CH₂)₃NH₂,
(b) a mixture of (i) a tertiary amine having the formula NR¹R²R³ where R¹ is a C₁-C₁₈ alkyl (preferably a C₈-C₁₈ alkyl) and R² and R³ are —(CH₂)₃NH₂, and (ii) one or more of sodium carbonate, a natural acid, and a natural base,
(c) erythorbic acid or a salt thereof,
(d) a mixture of (i) erythorbic acid or a salt thereof and (ii) one or more of a natural acid and a natural base, and
(e) a hydantoin or hydantoin blend.
   The aforementioned mixtures containing erythorbic acid (or a salt thereof) or a tertiary amine are synergistic.

According to one embodiment, after application to a substrate, the anti-allergenic agent and allergens may optionally be removed from the substrate by vacuuming, such as with a standard or steam vacuum.

Another embodiment is a liquid, solid, or aerosol formulation comprising an anti-allergenic effective amount of one or more anti-allergenic agents of the present invention. The liquid formulation typically includes a solvent, such as water. For example, the liquid formulation may be a cleaning solution (such as a hard surface cleaning solution) which includes an anti-allergenic effective amount of one or more anti-allergenic agents of the present invention. The solid formulation may be, for example, in the form of a powder or pellets. The aerosol formulation preferably includes a propellant.

Yet another embodiment is an aerosol can containing the aerosol formulation of the present invention.

Yet another embodiment is a method of controlling allergens in an environment or on a substrate by spraying an anti-allergenic effective amount of the aerosol formulation (such as from an aerosol can) into the environment or onto the substrate.

Yet another embodiment is a method of controlling allergens on a substrate by applying the liquid formulation (such as by brushing or spraying) or the solid formulation (such as by sprinkling) onto the substrate.

Yet another embodiment is a wipe (such as a pet wipe or baby wipe) containing an anti-allergenic effective amount of one or more anti-allergenic agents of the present invention.

### DETAILED DESCRIPTION OF THE INVENTION

In any identified embodiments, the term “about” means within 50%, preferably within 25%, and more preferably within 10% of a given value or range. Alternatively, the term “about” means within an acceptable standard error of the mean, when considered by one of ordinary skill in the art.

The term “allergens” as used herein includes, but is not limited to, all types of allergens (including household allergens), such as dust mites, dust mite allergens, cockroaches, cockroach allergens, dander (e.g., cat and dog dander), pollen, mold, and mildew. Mites are microscopic organisms frequently found in homes, the excrements to which people are often allergic

The term “controlling” as used herein refers to killing, inhibiting the growth of, immobilizing, denaturing and/or removing allergens from an environment, animal or substrate.

An “anti-allergenic effective amount” refers to an amount effective to kill, inhibit the growth of, immobilize, or remove allergens from an environment or a substrate. An anti-allergenic effective amount of any of the anti-allergenic agents described herein or mixtures thereof may be determined by methods known in the art, including the method described in Example 1 below.

The anti-allergenic agent and formulations of the present invention can be applied to an environment (such as the air in a room), animal, or a substrate by any method known in the art including, but not limited to, spraying, misting, dusting, brushing, wiping, or sprinkling the anti-allergenic agent in the environment or on the substrate.

The term “substrate” as used herein includes, but is not limited to, animals including their skin and fur; hard surfaces such as ceiling tiles, air vents, floors, counters, and tables; textile surfaces such as carpets, rugs, mattresses, bedding materials, upholstery, fabric toys, curtains, and window treatments; and other dust gathering surfaces. The anti-allergenic agents of the present invention may be applied, for example, in households, hotels, hospitals, schools, institutions, airplanes, cars, trains, and boats.

The term “anti-allergenic hyperbranched polymer” as used herein includes, but is not limited to, a polymer having a hyperbranched structure. Non-limiting examples of anti-allergenic hyperbranched polymers include Hybrane® 1690 and Hybrane® H-1500 (available from DSM of Heerlen, the Netherlands). Hybrane® polymers are made by polycondensation of cyclic anhydrides with diisopropanolamine. Hybrane® polymers have a hyperbranched structure, amide nitrogen atoms as branching points, and hydroxyl end groups in the base polymer.

The term “anti-allergenic amine” as used herein generally refers to primary, secondary, and tertiary amines, such as monoalkylamines, dialkyl amines, and trialkylamines, preferably where the alkyl group(s) have from 1 to 30 carbon atoms, and salts thereof. Preferred amines include those having the formula NR¹R²R³ and salts thereof, wherein R¹, R², and R³ are independently hydrogen, alkyl (such as C₁-C₂₀ alkyl), or aryl and are optionally substituted with a hydroxyl group (—OH) or an amino group (—NH₂). Preferably, at least one of R¹, R², and R³ is not hydrogen. Suitable primary amines include, but are not limited to those having the formula NH₂R¹ where R¹ is C₁-C₂₀ alkyl, which is optionally substituted with a hydroxyl group (—OH), e.g., monoethanolamine. The term “anti-allergenic tertiary amine” as used herein generally refers to compounds having the formula NR¹R²R³ where R¹, R², and R³ are as defined above but are not hydrogen. According to a preferred embodiment, R¹ is a C₁-C₁₈ alkyl (preferably C₈-C₁₈ alkyl) and R² and R³ are independently —(CH2)ₙNH₂ where n is 2 or 3. According to a more preferred embodiment, R¹ is a C₁-C₁₈ alkyl (preferably C₈-C₁₈ alkyl) and R² and R³ are —(CH₂)₃NH₂. Non-limiting examples of preferred tertiary amines include N,N-bis(3-aminopropyl) dodecylamine (N-(3-aminopropyl)-N-dodecylpropane-1,3-diamine), available as Lonzabac® 12 from Lonza Inc. of Fair Lawn, N.J.

Suitable hops extracts include, but are not limited to, hexahydro iso alpha acids, hexahydro beta acids, tetrahydro iso alpha acids, and mixtures thereof. For example, suitable hops extracts include hexahydrobeta acid, available from John I. Haas, Inc. of Washington, D.C.; and tetra hydro iso alpha acid (10% w/w) in aqueous alkaline solution available as Tetrahop Gold® from Botanix Ltd. of Eardiston and Paddock Wood, United Kingdom.

The term “anti-allergenic iodo derivatives” as used herein includes, but is not limited to,
(1) 3-iodo-2 propynyl derivatives such as 3-iodo-2-propynyl butyl carbamate (IPBC), 3-iodo-2 propynyl succinate and p-chlorophenyl-3-iodopropynyl formal;
(2) iodopropynyl carbamate compounds having the formula: 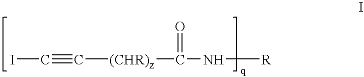
   wherein R is selected from the group consisting of hydrogen, and substituted and unsubstituted alkyl, aryl, and alkylaryl groups having from 1 to 20 carbon atoms; and q and z independently are integer from 1 to 3; and 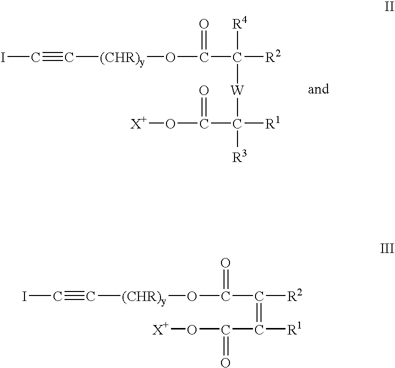
   wherein:
R¹ and R² are defined as R³ and R⁴ below or are joined to form a cycloalkyl, cycloalkenyl, aromatic or a heterocyclic ring containing an oxygen, nitrogen or sulfur atom or an alkoxy, amino, carboxyl, halo, hydroxyl, keto or a thiocarboxyl-substituted derivative thereof;
R³ and R⁴ are independently selected from
   (A) hydrogen, alkyl, cycloalkyl, alkenyl, cycloalkenyl, aryl, a heterocyclic ring containing an oxygen, nitrogen or sulfur atom, alkoxy, amino, carboxyl, halo, hydroxyl, keto or a thiocarboxyl; and
   (B) substituted derivatives of the alkyl, cycloalkyl, alkenyl, cycloalkenyl, aryl and the heterocyclic ring wherein the substitutions are alkyl, cycloalkyl, alkenyl, cycloalkenyl, aryl, alkoxy, amino, carboxyl, halo, hydroxyl, keto or a thiocarboxyl;
y is an integer from 0 to 16;
W is a single bond, oxygen, —N(R⁵)— or —(CR⁶R⁷)ₚ—;
R⁵ is hydrogen, alkyl, cycloalkyl, alkenyl, cycloalkenyl, aryl or a heterocyclic ring containing an oxygen, nitrogen or sulfur atom or a substituted derivative of alkyl, cycloalkyl, alkenyl, cycloalkenyl or aryl groups wherein the substitutions are alkyl, cycloalkyl, alkenyl, cycloalkenyl, aryl, alkoxy, amino, carboxyl, halo, hydroxyl, keto, or a thiocarboxyl;
R⁶ and R⁷ are defined as R³ and R⁴ above;
p is an integer from 1 to 12; and
X is hydrogen or a salt-forming cation such as an alkali metal, an alkaline earth metal, ammonium, tertiary ammonium, a quaternary ammonium, a biguanide or a polybiguanide.

The above definition of R⁵ includes, among other things, an aminoalkyl group.

The heterocyclic rings referred to in the above definitions may contain from 5 to 8 members, the alkyl or cycloalkyl groups from 1 to 18 atoms, the alkenyl or cycloalkenyl groups from 2 to 18 carbon atoms, and the aryl groups from 6 to 10 members.

In formula III, when R¹ and R² are hydrogen, the compound is a maleate. When R¹ and R² are joined together to form part of a six membered aromatic ring the compound is a phthalate. In formula II, when R¹, R², R³, and R⁴ are hydrogen and W is a single bond, the compound is a succinate. When R¹, R², R³, and R⁴ are hydrogen and W is an oxygen, the compound is a diglycolate. Other compounds include the mono-iodopropynyl esters of anhydrides such as ethylenediamine tetraacetic dianhydride, 3,3-dimethylglutaric anhydride, S-acetylmercaptosuccinic anhydride, dichloromaleic anhydride, 2-dodecen-1-yl succinic anhydride and cis-5-norbornene-endo-2,3-dicarboxylic anhydride. Where hydrophilicity is desired, the sodium salts may be used because of their extremely high water solubility. Preferred carboxylic acid anhydrides include, but are not limited to, succinic, itaconic, phthalic, tetrachlorophthalic, and diglycolic anhydride. Examples of such compounds are described in U.S. Pat. Nos. 4,844,891 and 5,073,570, both of whih are incorporated by reference. More preferably, the iodo derivative is 3-iodo-2-propynyl butyl carbamate (IPBC) or 3-iodo-2-propynyl N-butylcarbamate. The IPBC may be any grade of IPBC including, but not limited to, an essentially pure commercial grade IPBC in solid form and commercially available 6% and 10% grades in a solvent, available as Glycacil® 2000 from Lonza Inc. of Fair Lawn, N.J.

The term “anti-allergenic quaternary ammonium compound” refers to compounds having the formula (NR¹R²R³R⁴)⁺X⁻ wherein R¹, R², R³, and R⁴ are independently C₁-C₂₀ alkyl and more preferably C₁-C₁₂ alkyl and X is an anion. Preferred quaternary ammonium compounds include, but are not limited to, di-(C₈-C₁₂-alkyl)dimethyl quaternary ammonium compounds having the formula: 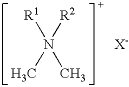
wherein R¹ and R² are independently C₈-C₁₂ alkyl groups and X is an anion. R¹ and R² may be the same or different. Suitable anions include, but are not limited to, chloride, carbonate, bicarbonate, hydroxide, and carboxylates. According to one preferred embodiment, R¹ and R² are C₁₀ alkyl groups (such as n-decyl).

The term “anti-allergenic quaternary ammonium compound” also refers to benzyl quaternary ammonium compounds, such as alkyl dimethyl benzyl ammonium chloride available (e.g. C₁₂-C₁₆ alkyl dimethyl benzyl ammonium chloride) as Barquat® 4250-Z from Lonza Inc. of Fair Lawn, N.J.; dialkyl quaternary ammonium compounds; alkyl dimethyl benzyl ammonium compounds (e.g., C₈-C₁₆ alkyl dimethyl benzyl ammonium compounds, C₈-C₁₂ alkyl dimethyl benzyl ammonium compounds, and their chloride salts); dialkyl dimethyl ammonium compounds (e.g., di-(C₈-C₁₆ alkyl) dimethyl ammonium compounds, di-(C₈-C₁₂ alkyl) dimethyl ammonium compounds, and their chloride salts) available as Bardac® 208M from Lonza Inc. of Fair Lawn, N.J.; didecyl dimethyl ammonium chloride, available as Bardac® 2280 from Lonza Inc. of Fair Lawn, N.J.; benzethonium salts, such as benzethonium chloride (diisobutylphenoxyethoxyethyl dimethyl benzyl ammonium chloride), available as Hyamine® 1622 from Lonza Inc. of Fair Lawn, N.J.; mixtures of alkyl dimethyl benzyl ammonium chloride and didecyl dimethyl ammonium chloride available as FMB® 1210-5 and FMB® 1210-8 from Lonza Inc. of Fair Lawn, N.J.; cetyl trimethyl ammonium chloride available as Carsoquat® CT-425 from Lonza Inc. of Fair Lawn, N.J.; and their carbonates and bicarbonates.

The term “anti-allergenic hydantoins” as used herein includes, but is not limited to, alkanoldialkyl hydantoins having the formula: 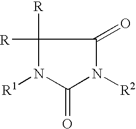
wherein each occurrence of R is independently hydrogen, a methyl group, an ethyl group, a propyl group, an alkyl group or an aryl group, and R¹ and R² are each independently hydrogen or (CH₂)OH, with the proviso that R¹ and R² cannot both be hydrogen. According to one embodiment, at least one of R¹ and R² is —(CH₂) OH. Preferred hydantoins include, but are not limited to, 1,3-dimethylol-5,5-dimethylhydantoin (DMDMH), 1-methylol-5,5-dimethylhydantoin/3-methylol-5,5-dimethylhydantoin (MMDMH), dimethylhydantoin (DMH), and mixtures thereof. A preferred blend contains DMDMH, MMDMH, and DMH, such as, for example, a blend of about 36% dimethylol dimethyl hydantoin (DMDMH), 29% monomethylol dimethylhydantoin (MMDMH), and 5% dimethyl hydantoin (DMH), and 30% water, available as Glydant® 2000 from Lonza Inc. of Fair Lawn, N.J.

Polyhexamethylenebiguanidine hydrochloride is available as Lonzabac® BG from Lonza Inc. of Fair Lawn, N.J.

Suitable anti-allergenic natural oils include, but are not limited to, hop oils, achillen fragrantissims, cinnamon oil, and neem oil.

Suitable anti-allergenic natural acids include, but are not limited to, lactic acid, citric acid, erythorbic acid, and a blend of acids such as lactic, citric, malic acids and tea tree oil. A preferred anti-allergenic natural acid is erythorbic acid.

Suitable anti-allergenic chelating agents include, but are not limited to, ethylene diamine tetra acetic acid (EDTA) and salts thereof (e.g., tetrasodium EDTA available as Versene 100® from Dow Europe S.A. of Horgen, Switzerland), and diammonium ethylene diamine tetraacetate.

The term “cyclic ketone acid” as used herein includes compounds that have a ring containing a carbonyl group. Suitable anti-allergenic cyclic ketone acids include, but are not limited to, those having the formula 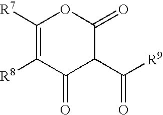
and salts thereof, wherein R⁷, R⁸, and R⁹ are independently C₁-C₁₀ alkyl, C₁-C₁₀ alkenyl, C₁-C₁₀ alkenyl, aryl, aryl substituted with halogen, or (C₁-C₁₀ alkyl)aryl. Preferably, R⁷, R⁸, and R⁹ are independently C₁-C₄ alkyl; or R⁷ and R⁸ form a 5-12 member ring.

Preferred cyclic ketone acids, include, but are not limited to, those having the formula 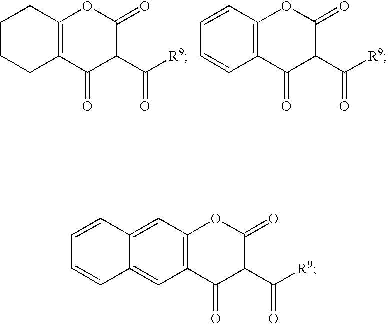
and salts thereof. A more preferred cyclic ketone acid is dehydroacetic acid and salts thereof (including hydrates thereof), such as sodium dehydroacetate (e.g., sodium dehydroacetate hydrate and sodium dehydroacetate monohydrate).

Anti-allergenic surfactants include, but are not limited to, nonyl phenol ethoxylate, amine oxides, amphoteric surfactants, ethylene oxide/propylene oxide block polymers, alkyl poly gluconates, fluorosurfactants, ethoxylated alcohols, and mixtures thereof. Suitable amphoteric surfactants include, but are not limited to, imidazoline amphoterics such as Amphoterge LF® from Lonza Inc. of Fair Lawn, N.J. Suitable oxides surfactants include, but are not limited to, lauryl dimethyl amine oxide available as Barlox® 12 from Lonza Inc. of Fair Lawn, N.J.; and decyl dimethyl amine oxide available as Barlox® 10S from Lonza Inc of Fair Lawn, N.J. Suitable ethoxylated alcohol surfactants include, but are not limited to glycerin 26 mole ethoxylate, lauric acid 23 mole ethoxylate, lauric acid 4 mole ethoxylate available as Ethosperse® from Lonza Inc. of Fair Lawn, N.J. Suitable surfactants also include, but are not limited to, silver iodide in a polymeric biguanide matrix available as Surfacine-D® from Lonza Inc. of Fair Lawn, N.J. Surfacine-D® as used herein includes a tetrafunctional block copolymer derived from the addition of ethylene oxide and propylene oxide to ethylenediamine such as Tetronic® 1107 available from BASF (USA), polyhexamethylene biguanide hydrochloride (20% solution), potassium iodide, silver nitrate, n-methyl-2-pyrrolidinone, propylene glycol n-butyl ether, Triton™ X-100 (polyoxyethylene octylphenol), ethyl alcohol, and water.

Suitable anti-allergenic organic phosphates include, but are not limited to, 1-hydroxyethane-1,1-diphosphonic acid, available as Unihib-106® from Lonza Inc. of Fair Lawn, N.J.

Another anti-allergenic agent of the present invention is a mixture of water, sodium lactate, lactic acid, glycerin, serine, sorbitol, TEA-Lactate, urea, sodium chloride, lauryl diethylenediaminoglycine, lauryl aminopropylglycine, allantoin, and SD alcohol 39-C (alcohol denat.), which is available as Hydroviton® from Dragoco Inc. of Totowa, N.J.

Preferred anti-allergenic agents and mixtures thereof include:

| Mixture | | | | |
|---|---|---|---|---|
| No. | Component A | Component B | Component C | Component D |
| 1 | sodium carbonate | an anti-allergenic | — | — |
| | | chelating agent, such | | |
| | | as tetrasodium | | |
| | | ethylene diamine | | |
| | | tetraacetate | | |
| 2 | sodium carbonate | an anti-allergenic | an anti-allergenic | — |
| | | chelating agent | surfactant (e.g. | |
| | | | sodium carbonate, | |
| | | | tetrasodium ethylene | |
| | | | diamine tetraacetate, | |
| | | | and an imidazoline | |
| | | | amphoteric | |
| | | | surfactant) | |
| 3 | sodium carbonate | anti-allergenic | an anti-allergenic | an anti- |
| | | chelating agent | tertiary amine | allergenic |
| | | | | surfactant |
| 4 | sodium carbonate | an anti allergenic | an anti-allergenic | — |
| | | tertiary amine | chelating agent | |
| 5 | an anti allergenic | dehydroacetic acid or | — | — |
| | tertiary amine, such | a salt thereof (e.g., | | |
| | as N,N-bis(3- | sodium | | |
| | aminopropyl) | dehydroacetate) | | |
| | dodecylamine | | | |
| 6 | dehydroacetic acid or | sodium carbonate | — | — |
| | a salt thereof | | | |
| 7 | a tertiary amine | — | — | — |
| | having the formula | | | |
| | NR¹R²R³ where R^{1 is} | | | |
| | a C₁-C₁₈ alkyl | | | |
| | (preferably a C₈-C₁₈ | | | |
| | alkyl) and R² and R³ | | | |
| | are —(CH₂)₃NH₂ | | | |
| 8 | a tertiary amine | sodium carbonate | — | — |
| | having the formula | | | |
| | NR¹R²R³ where R¹ is | | | |
| | a C₁-C₁₈ alkyl | | | |
| | (preferably a C₈-C₁₈ | | | |
| | alkyl) and R² and R³ | | | |
| | are —(CH₂)₃NH₂ | | | |
| 9 | a tertiary amine | natural acid or natural | — | — |
| | having the formula | base | | |
| | NR¹R²R³ where R¹ is | | | |
| | a C₁-C₁₈ alkyl | | | |
| | (preferably a C₈-C₁₈ | | | |
| | alkyl) and R² and R³ | | | |
| | are —(CH₂)₃NH₂ | | | |
| 10 | erythorbic acid or | — | — | — |
| | salt thereof | | | |
| 11 | erythorbic acid or | natural acids or | — | — |
| | salt thereof | natural bases | | |

A preferred mixture 3 is sodium carbonate, tetrasodium ethylene diamine tetraacetate, N,N-bis(3-aminopropyl) dodecylamine, and an imidazoline amphoteric surfactant.

A preferred mixture 4 is sodium carbonate, N,N-bis(3-aminopropyl) dodecylamine, and tetrasodium ethylene diamine tetraacetate.

In mixtures 7-9, the tertiary amine is preferably N,N-bis(3-aminopropyl) dodecylamine. In mixture 8, the weight ratio of the tertiary amine to sodium carbonate preferably ranges from about 0.01:3 to about 5:0.01 and more preferably from about 0.05:3 to about 1:0.05. In mixture 9, the weight ratio of the tertiary amine to natural acid or natural base preferably ranges from about 0.01:5 to about 5:0.01 and more preferably from about 0.1:2 about 3:0.1.

In mixture 11, the weight ratio of the erythorbic acid or a salt thereof to natural acid or natural base preferably ranges from about 0.01:5 to about 5:0.01 and more preferably from about 0.1:2 to about 3:0.1.

Formulations containing the anti-allergenic agents or the anti-allergenic agent mixtures above typically include from about 0.01% to about 10% by weight of the anti-allergenic agents (in total). According to one embodiment, the formulations include from about 0.1% to about 5% by weight of the anti-allergenic agents (in total), and preferably from about 0.2 to 2% by weight of the anti-allergenic agents (in total).

The preferred mixtures identified above are preferably included as use dilutions in the formulations of the present invention at the weight percentages specified in the table below.

| Mixture | | |
|---|---|---|
| No. | Preferred | More Preferred |
| 1 | about 0.01 to about 5% component A | about 0.05 to about 3% component A |
| | about 0.01 to about 5% component B | about 0.05 to about 3% component B |
| 2 | about 0.01 to about 5% component A | about 0.05 to about 3% component A |
| | about 0.01 to about 5% component B | about 0.05 to about 3% component B |
| | about 0.01 to about 5% component C | about 0.05 to about 3% component C |
| 3 | about 0.01 to about 5% component A | about 0.05 to about 3% component A |
| | about 0.01 to about 5% component B | about 0.05 to about 3% component B |
| | about 0.01 to about 5% component C | about 0.05 to about 3% component C |
| | about 0.01 to about 5% component D | about 0.05 to about 3% component D |
| 4 | about 0.01 to about 5% component A | about 0.05 to about 3% component A |
| | about 0.01 to about 5% component B | about 0.05 to about 3% component B |
| | about 0.01 to about 5% component C | about 0.05 to about 3% component C |
| 5 | about 0.01 to about 5% component A | about 0.05 to about 3% component A |
| | about 0.01 to about 5% component B | about 0.05 to about 3% component B |
| 6 | about 0.01 to about 5% component A | about 0.05 to about 3% component A |
| | about 0.01 to about 5% component B | about 0.05 to about 3% component B |
| 7 | about 0.01 to about 10% component A | about 0.1 to about 5% component A |
| 8 | about 0.01 to about 5% component A | about 0.05 to about 3% component A |
| | about 0.01 to about 3% component B | about 0.05 to about 1% component B |
| 9 | about 0.01 to about 5% component A | about 0.1 to about 3% component A |
| | about 0.01 to about 5% component B | about 0.1 to about 2% component B |
| 10 | about 0.01 to about 10% component A | about 0.1 to about 5% component A |
| 11 | about 0.01 to about 5% component A | about 0.1 to about 3% component A |
| | about 0.01 to about 5% component B | about 0.1 to about 2% component B |

Optionally, further removal of allergens from the substrate after application of the anti-allergenic agent may be accomplished by vacuuming the substrate. Vacuuming the substrate advantageously improves the allergen environment and can achieve about an 99.99% allergen free substrate. Any type of vacuum may be used, including a steam vacuum.

The following examples are intended to describe the present invention without limitation.

### EXAMPLE 1

Several samples were tested using a standard Enzyme Linked Immunosorbent Assay, (ELISA). The ELISA method for Der p 1 Allergen was utilized. Der p 1 allergen is a standardized allergen. The ELISA method provides quantitative results correlating to the level of allergen recovered in the test.

In the ELISA method for Der p 1 Allergen, the coating antibody is Anti-Der p1 mAb 5H8, supplied as a HPLC purified stock solution at 2 mg/ml in phosphate buffered saline. The coating antibody was diluted in coating buffer (12 μl in 12 ml). 100 μl of the diluted coating antibody was dispensed in each well. The plate was held overnight at 4° C. Then the coating antibody was dumped from the plate and the plate was blotted dry. The well was washed 3 times with PBS-T and blotted dry. Next, 100 μl per well of blocking buffer (BSA PBS-T) was added and incubated for 30 minutes at room temperature. A standard curve (doubling dilutions between 250-0.5 ng/ml) was prepared. The experimental samples were prepared. “Controls” are actives exposed to the plate before addition of Ag. The exposure time was 1 hour. The Ag level used in the plate was 62.5 ng/ml as is for the Ag level in the active. “Samples” are Actives exposed to Ag before addition to the plate. Exposure time was 10 minutes before incubation in the plate for 1 hour. Actives were used in 1:1 ratio with 125 ng/ml Ag (yield 62.5 ng/ml Ag in test plate).

The plate was then washed, filled with standards, blanks, controls and samples and incubated for 1 hour. The detecting antibody was prepared. Next, the plate was washed and the detecting antibody was added to each well. The conjugate was prepared. The plate was washed and the conjugate was added. Just before washing the conjugate from the plate, H₂O₂ was added to the substrate solution. The plate was washed and the substrate was added. The plate was read when the absorbance of 250 ng/ml antigen standard (at 405 nm) reached 2.0-2.4.

Table 1 summarizes the efficacy of various anti-allergenic agents tested by this method. The active levels in the table are listed in ppm and are diluted in half by antigen during testing.

TABLE 1

| Sample | Diluent | Active (ppm) | % Reduction |
|---|---|---|---|
| N,N-bis(3-aminopropyl) | water | 50,000 | 100 |
| dodecylamine | | | |
| N,N-bis(3-aminopropyl) | water | 15,000 | 99 |
| dodecylamine | | | |
| lactic acid | water | 3,500 | 95 |
| citric acid | water | 5,000 | 100 |
| erythorbic acid | water | 10,000 | 96 |
| ascorbic acid | water | 10,000 | 91 |
| Ethosperse ™ - | None | 500,000 | 92 |
| ethoxylated alcohol | | | |
| tetrasodium ethylene | water | 15,000 | 91 |
| diamine tetraacetate | | | |
| dehydroacetic acid | ethoxylated | 2,500 | 100 |
| | alcohol | | |
| sodium carbonate | water | 2,500 | 100 |
| N,N-bis(3-aminopropyl) | water | 1,500 + 3,000 | 96 |
| dodecylamine + sodium | | | |
| carbonate | | | |
| Erythorbic Acid + Decyl | water | 15,000 + 1,000 | 99 |
| dimethyl amine oxide | | | |
| Dehydroacetic acid | water | 5,000 + 1,100 | 79 |
| sodium salt + sodium | | | |
| carbonate | | | |
| N,N-bis(3-aminopropyl) | water | 7,500 + 5,000 | 67 |
| dodecylamine + | | | |
| didecyl | | | |
| dimethylammonium | | | |
| chloride | | | |
| sodium carbonate + | water | 3,000 + 1,500 | 95 |
| didecyl dimethyl | | | |
| ammonium chloride | | | |

All patents, applications, articles, publications, and test methods mentioned above are hereby incorporated by reference.

1. A method of controlling allergens comprising the step of applying to the allergens an anti-allergenic effective amount of at least one anti-allergenic agent selected from
(a) a tertiary amine having the formula NR¹R²R³ where R¹ is a C₁-C₁₈ alkyl and R² and R³ are —(CH₂)₃NH₂,
(b) a mixture of (i) a tertiary amine having the formula NR¹R²R³ where R¹, R², and R³ are as defined above, and (ii) one or more of sodium carbonate, a natural acid, and a natural base,
(c) erythorbic acid or a salt thereof,
(d) a mixture of (i) erythorbic acid or a salt thereof and (ii) one or more of a natural acid and a natural base, and
(e) a hydantoin or hydantoin blend.

2. The method of claim 1, wherein the anti-allergenic agent comprises a tertiary amine having the formula NR¹R²R³.

3. The method of claim 1, wherein the anti-allergenic agent comprises a tertiary amine having the formula NR¹R²R³ and sodium carbonate.

4. The method of claim 3, wherein the weight ratio of the tertiary amine to sodium carbonate is from about 0.01:3 to about 5:0.01.

5. The method of claim 1, wherein the anti-allergenic agent comprises a tertiary amine having the formula NR¹R²R³ and at least one natural acid.

6. The method of claim 5, wherein the weight ratio of the tertiary amine to natural acid is from about 0.01:5 to about 5:0.01.

7. The method of claim 1, wherein the anti-allergenic agent comprises a tertiary amine having the formula NR¹R²R³ and at least one natural base.

8. The method of claim 7, wherein the weight ratio of the tertiary amine to natural base is from about 0.01:5 to about 5:0.01.

9. The method of claim 1, wherein the anti-allergenic agent comprises erythorbic acid or a salt thereof.

10. The method of claim 1, wherein the anti-allergenic agent comprises erythorbic acid or a salt thereof and at least one natural acid.

11. The method of claim 10, wherein the weight ratio of erythorbic acid or salt thereof to natural acid is from about 0.01:5 to about 5:0.01.

12. The method of claim 1, wherein the anti-allergenic agent comprises erythorbic acid or a salt thereof and at least one natural base.

13. The method of claim 12, wherein the weight ratio of erythorbic acid or salt thereof to natural base is from about 0.01:5 to about 5:0.01.

14. An anti-allergenic formulation comprising (a) a tertiary amine having the formula NR¹R²R³ where R¹ is a C₁-C₁₈ alkyl and R² and R³ are —(CH₂)₃NH₂, and (b) sodium carbonate.

15. The formulation of claim 14, wherein the weight ratio of the tertiary amine to sodium carbonate is from about 0.01:3 to about 5:0.01.

16. An anti-allergenic formulation comprising (a) a tertiary amine having the formula NR¹R²R³ where R¹ is a C₁-C₁₈ alkyl and R² and R³ are —(CH₂)₃NH₂, and (b) at least one natural acid.

17. The formulation of claim 16, wherein the weight ratio of the tertiary amine to natural acid is from about 0.01:5 to about 5:0.01.

18. An anti-allergenic formulation comprising (a) a tertiary amine having the formula NR¹R²R³ where R¹ is a C₁-C₁₈ alkyl and R² and R³ are —(CH₂)₃NH₂, and (b) at least one natural base.

19. The formulation of claim 18, wherein the weight ratio of the tertiary amine to natural base is from about 0.01:5 to about 5:0.01.

20. An anti-allergenic formulation comprising (a) erythorbic acid or a salt thereof and (b) at least one natural acid.

21. The formulation of claim 20, wherein the weight ratio of the erythorbic acid or a salt thereof to natural acid is from about 0.01:5 to about 5:0.01.

22. An anti-allergenic formulation comprising (a) erythorbic acid or a salt thereof and (b) at least one natural acid.

23. The formulation of claim 22, wherein the weight ratio of the erythorbic acid or a salt thereof to natural base is from about 0.01:5 to about 5:0.01.

24. A household formulation comprising an anti-allergenic effective amount of at least one anti-allergenic agent selected from
(a) a tertiary amine having the formula NR¹R²R³ where R¹ is a C₁-C₁₈ alkyl and R² and R³ are —(CH₂)₃NH₂,
(b) a mixture of (i) a tertiary amine having the formula NR¹R²R³ where R¹, R², and R³ are as defined above, and (ii) one or more of sodium carbonate, a natural acid, and a natural base,
(c) erythorbic acid or a salt thereof,
(d) a mixture of (i) erythorbic acid or a salt thereof and (ii) one or more of a natural acid and a natural base, and
(e) a hydantoin or hydantoin blend.

25. The formulation of claim 24, wherein the formulation is a liquid, solid, or aerosol.

26. A wipe comprising an anti-allergenic effective amount of at least one anti-allergenic agent selected from
(a) a tertiary amine having the formula NR¹R²R³ where R¹ is a C₁-C₁₈ alkyl and R² and R³ are —(CH₂)₃NH₂,
(b) a mixture of (i) a tertiary amine having the formula NR¹R²R³ where R¹, R², and R³ are as defined above, and (ii) one or more of sodium carbonate, a natural acid, and a natural base,
(c) erythorbic acid or a salt thereof,
(d) a mixture of (i) erythorbic acid or a salt thereof and (ii) one or more of a natural acid and a natural base, and
(e) a hydantoin or hydantoin blend.

27. A method of controlling allergens on a substrate comprising the step of applying to the substrate an anti-allergenic effective amount of at least one anti-allergenic agent selected from
(a) a tertiary amine having the formula NR¹R²R³ where R¹ is a C₁-C₁₈ alkyl and R² and R³ are —(CH₂)₃NH₂,
(b) a mixture of (i) a tertiary amine having the formula NR¹R²R³ where R¹, R², and R³ are as defined above, and (ii) one or more of sodium carbonate, a natural acid, and a natural base,
(c) erythorbic acid or a salt thereof,
(d) a mixture of (i) erythorbic acid or a salt thereof and (ii) one or more of a natural acid and a natural base, and
(e) a hydantoin or hydantoin blend.

28. The method of claim 27, further comprising the step of vacuuming the substrate.
